(19) United States
(12) Patent Application Publication
(10) Pub. No.: US 2014/0163263 A1
(43) Pub. Date: Jun. 12, 2014

(54) Group VIII Metal Hydrogenolysis Catalysts Having Low Selectivity to Ethers

(71) Applicant: CELANESE INTERNATIONAL CORPORATION, Irving, TX (US)

(72) Inventors: Radmila Wollrab, Pasadena, TX (US); Zhenhua Zhou, Houston, TX (US); James H. Zink, League City, TX (US); Victor J. Johnston, Houston, TX (US); Heiko Weiner, Pasadena, TX (US)

(73) Assignee: CELANESE INTERNATIONAL CORPORATION, Irving, TX (US)

(21) Appl. No.: 13/709,404

(22) Filed: Dec. 10, 2012

(51) Int. Cl. C07C 29/149

(52) U.S. Cl. CPC C07C 29/149
USPC 568/885

(57) ABSTRACT

Group VIII metal containing catalysts used in processes for producing ethanol from ethyl acetate by reacting the ethyl acetate with hydrogenation. The Group VIII metal containing catalyst has a selectivity to ether, especially diethyl ether, that is very low. The process may be integrated with an ethyl acetate production process, such as esterification, hydrogenation, or dehydrogenation.

### FIELD OF THE INVENTION

The present invention relates generally to processes for alcohol production from ethyl acetate using a Group VIII metal containing catalyst, and in particular, to processes for producing ethanol from ethyl acetate with low selectivity to diethyl ether.

### BACKGROUND OF THE INVENTION

Ethanol for industrial use is conventionally produced from petrochemical feed stocks, such as oil, natural gas, or coal, from feed stock intermediates, such as syngas, or from starchy materials or cellulosic materials, such as corn or sugar cane. Conventional methods for producing ethanol from petrochemical feed stocks, as well as from cellulose materials, include the acid-catalyzed hydration of ethylene, methanol homologation, direct alcohol synthesis, and Fischer-Tropsch synthesis. Instability in petrochemical feed stock prices contributes to fluctuations in the cost of conventionally produced ethanol, making the need for alternative sources of ethanol production all the greater when feed stock prices rise. Starchy materials, as well as cellulosic material, are converted to ethanol by fermentation. However, fermentation is typically used for consumer production of ethanol, which is suitable for fuels or human consumption. In addition, fermentation of starchy or cellulosic materials competes with food sources and places restraints on the amount of ethanol that can be produced for industrial use.

Ethanol production via the reduction of alkanoic acids and/or other carbonyl group-containing compounds, including esters, has been widely studied, and a variety of combinations of catalysts, supports, and operating conditions have been mentioned in the literature. Copper-iron catalysts for hydrogenolyzing esters to alcohols are described in U.S. Pat. No. 5,198,592. A hydrogenolysis catalyst comprising nickel, tin, germanium and/or lead is described in U.S. Pat. No. 4,628,130. A rhodium hydrogenolysis catalyst that also contains tin, germanium and/or lead is described in U.S. Pat. No. 4,456,775.

Several processes that produce ethanol from acetates, including methyl acetate and ethyl acetate, are known in the literature.

WO8303409 describes producing ethanol by carbonylation of methanol by reaction with carbon monoxide in the presence of a carbonylation catalyst to form acetic acid which is then converted to an acetate ester followed by hydrogenolysis of the acetate ester formed to give ethanol or a mixture of ethanol and another alcohol which can be separated by distillation. Preferably the other alcohol or part of the ethanol recovered from the hydrogenolysis step is recycled for further esterification. Carbonylation can be effected using a CO/H₂ mixture and hydrogenolysis can similarly be conducted in the presence of carbon monoxide, leading to the possibility of circulating gas between the carbonylation and hydrogenolysis zones with synthesis gas, preferably a 2:1 H₂:CO molar mixture being used as makeup gas.

WO2009009320 describes an indirect route for producing ethanol. Carbohydrates are fermented under homoacidogenic conditions to form acetic acid. The acetic acid is esterified with a primary alcohol having at least 4 carbon atoms and hydrogenating the ester to form ethanol.

US Pub. No. 2011004034 describes a continuous process for the production of ethanol from a carbonaceous feedstock. The carbonaceous feedstock is first converted to synthesis gas which is then converted to ethanoic acid, which is then esterified and which is then hydrogenated to produce ethanol.

US Pub. No. 2011046421 describes a process for producing ethanol comprising converting carbonaceous feedstock to syngas and converting the syngas to methanol. Methanol is carbonylated to ethanoic acid, which is then subjected to a two stage hydrogenation process. First the ethanoic acid is converted to ethyl ethanoate followed by a secondary hydrogenation to ethanol.

U.S. Pat. No. 7,964,379 describes a process for producing acetic acid intermediate from carbohydrates, such as corn, using enzymatic milling and fermentation steps. The acetic acid intermediate is acidified with calcium carbonate and the acetic acid is esterified to produce esters. Ethanol is produced by a hydrogenolysis reaction of the ester.

U.S. Pat. No. 5,414,161 describes a process for producing ethanol by a gas phase carbonylation of methanol with carbon monoxide followed by a hydrogenation. The carbonylation produces acetic acid and methyl acetate, which are separated and the methyl acetate is hydrogenated to produce ethanol in the presence of a copper-containing catalyst.

U.S. Pat. No. 4,497,967 describes a process for producing ethanol from methanol by first esterifying the methanol with acetic acid. The methyl acetate is carbonylated to produce acetic anhydride which is then reacted with one or more aliphatic alcohols to produce acetates. The acetates are hydrogenated to produce ethanol. The one or more aliphatic alcohols formed during hydrogenation are returned to the acetic anhydride esterification reaction.

U.S. Pat. No. 4,454,358 describes a process for producing ethanol from methanol. Methanol is carbonylated to produce methyl acetate and acetic acid. The methyl acetate is recovered and hydrogenated to produce methanol and ethanol. Ethanol is recovered by separating the methanol/ethanol mixture. The separated methanol is returned to the carbonylation process.

The need remains for improved processes for efficient ethanol production by reducing esters on a commercially feasible scale.

### SUMMARY OF THE INVENTION

In a first embodiment, the present invention is directed to a method of producing ethanol comprising contacting a feed stream comprising ethyl acetate, i.e. from 90 to 99.9 wt. % ethyl acetate, with hydrogen in a reactor in the presence of a catalyst to produce a crude ethanol product that comprises less than 0.5 wt. % ether compounds and preferably from 0.0001 to 0.5 wt. % diethyl ether, wherein the catalyst comprises tin, a Group VIII metal selected from the group consisting of palladium, platinum, and combinations thereof, and a support that comprises calcium, magnesium, tungsten, or molybdenum, provided that when the support comprises tungsten and/or molybdenum, the catalyst further comprises cobalt and/or the support further comprises cobalt and/or tin. Selectivity to ether compounds is less than 1%, selectivity to ethanol is at least 80%, and conversion of ethyl acetate is at least 25%.

The catalyst may comprise a support material selected from the group consisting of silica, pyrogenic silica, and high purity silica. The support and support material may be substantially free of alumina. In one aspect, the support comprises from 1 to 25 wt. % calcium or magnesium, based on the total weight of the catalyst. In some embodiments, the support comprises calcium oxide, calcium silicate, calcium metasilicate, magnesium oxide, magnesium silicate, or magnesium metasilicate. In another aspect, the support comprises from 5 to 30 wt. % tungsten or molybdenum, based on the total weight of the catalyst, and cobalt and/or tin on the support. In this aspect, the support comprises tungsten oxide, cobalt tungstate, molybdenum oxide, cobalt molybdate, or combinations thereof. In either aspect, the support is substantially free of tin tungstate.

In one embodiment, the catalyst may comprise from 0.1 to 7.5 wt. % tin, based on the total weight of the catalyst. The catalyst may also comprise from 0.1 to 3 wt. % Group VIII metal, based on the total weight of the catalyst.

In one embodiment, the process may further comprise separating ethyl acetate, acetic acid, and acetaldehyde from the crude ethanol mixture and recycling the separated compounds to the feed stream.

In a second embodiment, the present invention is directed to a method of producing ethanol comprising directing acetic acid and ethanol in a molar ratio of greater than 1.01:1 to a first reaction zone to produce a feed stream; and reacting at least a portion of the feed stream with hydrogen in a second reaction zone to produce a crude ethanol product comprising less than 0.5 wt. % diethyl ether, wherein the second reaction zone contains a catalyst that comprises tin, a Group VIII metal selected from the group consisting of palladium and platinum, and a support that comprises calcium, magnesium, tungsten, or molybdenum, provided that when the support comprises tungsten and/or molybdenum, the catalyst further comprises cobalt and/or the support further comprises cobalt and/or tin.

In a third embodiment, the present invention is directed to a method of producing ethanol comprising esterifying acetic acid and ethanol in a first reaction zone to produce a feed stream and reacting at least a portion of the feed stream with hydrogen in a second reaction zone to produce a crude ethanol product comprising ethyl acetate, ethanol, and preferably less than 0.5 wt. % diethyl ether, wherein the second reaction zone contains a catalyst that comprises tin, a Group VIII metal selected from the group consisting of palladium and platinum, and a support that comprises calcium, magnesium, tungsten, or molybdenum, provided that when the support comprises tungsten and/or molybdenum, the catalyst further comprises cobalt and/or the support further comprises cobalt and/or tin. Optionally the crude ethanol product may comprise at least one alcohol having at least 4 carbon atoms. The process further comprises separating at least a portion of the crude ethanol product in a first distillation column to yield a first distillate comprising ethyl acetate and a first residue comprising ethanol, and separating at least a portion of the first residue in a second distillation column to yield an ethanol side stream. A second residue comprising the at least one alcohol having at least 4 carbon atoms may also be withdrawn.

### BRIEF DESCRIPTION OF DRAWINGS

The invention is described in detail below with reference to the appended drawings, wherein like numerals designate similar parts.

FIG. 1 is a schematic diagram of ethanol production process that directly feeds an organic phase of the esterification product produced by vapor esterification to the hydrogenolysis zone in accordance with one embodiment of the present invention.

### DETAILED DESCRIPTION OF THE INVENTION

### Introduction

The present invention relates to processes for producing ethanol from ethyl acetate by reacting the ethyl acetate with hydrogen in the presence of a Group VIII metal containing catalyst. In particular, the Group VIII metal containing catalyst of the present invention has a selectivity to ether, especially diethyl ether, that is low. A low selectivity to ether will produce a crude ethanol product having a low ether concentration. Ethers are not readily converted to ethanol and thus must be purged from the system. Purging ethers represents an adverse loss in ethanol production, and decreases raw material efficiency.

The Group VIII metal containing catalyst of the present invention is composed to be selective to ethanol with sufficient conversion of ethyl acetate. In one embodiment, the selectivity to the ether is less than 1%, e.g., less than 0.5%. For purposes of the present application, the term “ether” or “ethers” may refer to a dialkyl ether, such as diethyl ether, dimethyl ether, dipropyl ether, and mixtures thereof.

Low selectivities may produce a crude ethanol product having less than 0.5 wt. % ether, e.g., less than 0.3 wt. % or less than 0.1 wt. %. In one embodiment, the crude ethanol product may have an ether concentration from 0 to 0.5 wt. %, e.g., from 0.0001 to 0.5 wt. % or from 0.05 to 0.3 wt. %. Some catalysts may not be selective to ethers and may produce a crude ethanol product that is substantially free of ethers.

Selectivity to other impurities, such as acetaldehyde, acetic acid, and diethyl acetal, may increase the separation requirements, but do not represent an adverse loss in ethanol production. These impurities, unlike ether, may be converted to ethanol either using the same Group VIII metal containing catalyst or a suitable hydrogenation catalyst.

Without being bound by theory, ethers may be formed by dehydrating ethanol in the presence of an acidic catalyst. The Group VIII metal containing catalysts of the present invention, although acidic, may contain more Brønsted acidic sites that reduce formation of ethers. For example, the Group VIII metal containing catalysts of the present invention are substantially free of alumina. In another embodiment, when the Group VIII metal containing catalysts also contains tungsten and/or molybdenum, the catalyst further contains a sufficient amount of cobalt and/or a support having cobalt and/or tin to reduce the acidity of the catalyst. Although acetic acid may be formed during the reaction, it does not appear to be sufficient to drive the dehydration of ethanol to diethyl ether. Also, ether formation through dehydrating ethanol may be promoted at lower temperatures of less than 180° C.

### Catalyst

The Group VIII metal containing catalyst of the present invention comprises tin, and a Group VIII metal selected from the group consisting of palladium, platinum, and combinations thereof. The catalyst may include a support material selected from the group consisting of silica, pyrogenic silica, and high purity silica. In addition, the support may comprise calcium, magnesium, tungsten, or molybdenum, provided that when the support comprises tungsten and/or molybdenum, the catalyst further comprises cobalt and/or the support further comprises cobalt and/or tin. In one preferred embodiment, the catalyst may comprise cobalt as a promoter metal and the support may comprise cobalt and tin when the support comprises either tungsten or molybdenum.

In one embodiment, the catalyst of the present invention comprises tin in an amount from 0.1 to 7.5 wt. %, based on the total weight of the catalyst, e.g., from 0.5 to 5 wt. %. Although tin may be present as an oxide, it is preferred that the catalyst is substantially free of tin tungstate. The Group VIII metal on the catalyst may be present in an amount from 0.1 to 3 wt. %, based on the total weight of the catalyst, e.g., from 0.1 to 1.5 wt. %. The metal ratios of tin to Group VIII metal may vary from 10:1 to 1:10, e.g., from 4:1 to 1:4, from 2:1 to 1:2, from 1.5:1 to 1:1.5 or from 1.1:1 to 1:1.1.

The catalyst may also comprise a promoter metal that is used in addition to the tin and Group VIII metal. The promoter metal may be selected from the group consisting of cobalt, ruthenium, rhenium, nickel, titanium, iron, cesium, and chromium. A promoter metal may be used, for example, when the support contains tungsten or molybdenum. When present, the total weight of the promoter metal preferably is from 0.05 to 7.5 wt. %, based on the total weight of the catalyst e.g., from 0.1 to 4 wt. %. In some embodiments, the catalyst for converting ethyl acetate to ethanol may be substantially free of copper and zinc.

The support of the catalyst may be the majority component comprising from 90 to 99.9 wt. %, based on the total weight of the catalyst, e.g., from 93 to 99 wt. %. As indicated above, the support comprises a support material selected from the group consisting of silica, pyrogenic silica, and high purity silica. Preferably, the support material is substantially free of alumina.

In preferred embodiments, the support material comprises a silicaceous support material, e.g., silica, having a surface area of at least 50 m²/g, e.g., at least 100 m²/g, or at least 150 m²/g. In terms of ranges, the silicaceous support material preferably has a surface area from 50 to 600 m²/g, e.g., from 100 to 500 m²/g or from 100 to 300 m²/g. High surface area silica, as used throughout the application, refers to silica having a surface area of at least 250 m²/g. For purposes of the present specification, surface area refers to BET nitrogen surface area, meaning the surface area as determined by ASTM D6556-04, the entirety of which is incorporated herein by reference.

The preferred silicaceous support material also preferably has an average pore diameter from 5 to 100 nm, e.g., from 5 to 30 nm, from 5 to 25 nm or from 5 to 10 nm, as determined by mercury intrusion porosimetry, and an average pore volume from 0.5 to 2.0 cm³/g, e.g., from 0.7 to 1.5 cm³/g or from 0.8 to 1.3 cm³/g, as determined by mercury intrusion porosimetry.

The morphology of the support material, and hence of the resulting catalyst composition, may vary widely. In some exemplary embodiments, the morphology of the support material and/or of the catalyst composition may be pellets, extrudates, spheres, spray dried microspheres, rings, pentarings, trilobes, quadrilobes, multi-lobal shapes, or flakes although cylindrical pellets are preferred. Preferably, the silicaceous support material has a morphology that allows for a packing density from 0.1 to 1.0 g/cm³, e.g., from 0.2 to 0.9 g/cm³ or from 0.3 to 0.8 g/cm³. In terms of size, the silica support material preferably has an average particle size, meaning the average diameter for spherical particles or average longest dimension for non-spherical particles, from 0.01 to 1.0 cm, e.g., from 0.1 to 0.7 cm or from 0.2 to 0.5 cm. Since the precious metal and the one or more active metals that are disposed on the support are generally in the form of very small metal (or metal oxide) particles or crystallites relative to the size of the support, these metals should not substantially impact the size of the overall catalyst particles. Thus, the above particle sizes generally apply to both the size of the support as well as to the final catalyst particles, although the catalyst particles are preferably processed to form much larger catalyst particles, e.g., extruded to form catalyst pellets.

As indicated, the support includes one of calcium, magnesium, tungsten, or molybdenum, which may be referred to as a support modifier. In one embodiment, the support modifier may be a basic modifier that has a low volatility or no volatility and is based on calcium or magnesium. For example, the support may include one or more of calcium oxide, calcium silicate, calcium metasilicate, magnesium oxide, magnesium silicate, or magnesium metasilicate. The support may comprise from 1 to 25 wt. % calcium or magnesium, based on the total weight of the catalyst, e.g., from 3 to 15 wt. %.

In another embodiment, the support modifier may be based on tungsten or molybdenum. The support may comprise from 5 to 30 wt. % tungsten or molybdenum, based on the total weight of the catalyst, e.g., from 5 to 20 wt. %. When based on tungsten or molybdenum, the support may include cobalt, tin, or both. Cobalt and/or tin is added to the support with the tungsten or molybdenum and calcined prior to adding the Group VIII metal and tin. The support may comprise from 0.1 to 7.5 wt. % cobalt and/or tin, based on the total weight of the support, when added with tungsten or molybdenum.

Reduced tungsten oxides or molybdenum oxides may also be employed, such as, for example, one or more of WO₃, W₂₀O₅₈, WO₂, W₄₉O₁₁₉, W₅₀O₁₄₈, W₁₈O₄₉, MO₉O₂₆, MO₈O₂₃, MO₅O₁₄, MO₁₇O₄₇, MO₄O₁₁, or MoO₂. In one embodiment, the tungsten oxide may be cubic tungsten oxide (H_{0.5}WO₃). In addition, when cobalt and/or tin is used, the support may comprise tungsten oxide, cobalt tungstate, molybdenum oxide, cobalt molybdate, or combinations thereof. Preferably, the support is substantially free and does not comprise tin tungstate.

Exemplary catalysts for the present invention may include a silica support containing from 1 to 25 wt. % calcium or magnesium, from 0.1 to 7.5 wt. % tin, and from 0.1 to 3 wt. % Group VIII metal. Another exemplary catalyst for the present invention may include a silica support containing from 5 to 30 wt. % tungsten or molybdenum, and cobalt and/or tin on the support in the amount from 0.1 to 7.5 wt. %, and from 0.1 to 7.5 wt. % tin, from 0.1 to 3 wt. % Group VIII metal, and optionally from 0.05 to 7.5 wt. % of a promoter metal, such as cobalt.

In one embodiment, the catalyst may be prepared by impregnation. A calcium, magnesium, tungsten, or molybdenum precursor is added to the support material and dried and calcined. After calcination, a suitable tin precursor and Group VIII metal precursor may be added through impregnation. Catalyst preparation methods are further described in U.S. Pub. No. 2010/012114, the entire contents and disclosure of which are hereby incorporated by reference.

### Hydrogenolysis Reaction Conditions

Ethyl acetate and hydrogen are introduced into a hydrogenolysis reactor containing the Group VIII metal containing catalyst at a molar ratio of hydrogen to ethyl acetate that is greater than 2:1, e.g. greater than 4:1, or greater than 12:1. In terms of ranges, the molar ratio may be from 2:1 to 100:1, e.g., 4:1 to 50:1, or from 12:1 to 20:1. Without being bound by theory, higher molar ratios of hydrogen to ethyl acetate, preferably from 8:1 to 20:1, are believed to result in higher conversion and/or selectivity to ethanol.

The hydrogenolysis reactor may comprise any suitable type of reactor, such as a fixed bed reactor or a fluidized bed reactor. Hydrogenolysis reactions are exothermic and in many embodiments, an adiabatic reactor may be used for the hydrogenolysis reactor. Adiabatic reactors have little or no need for internal plumbing through the reaction zone to add or remove heat. In other embodiments, a radial flow reactor or reactors may be employed, or a series of reactors may be employed with or without heat exchange, quenching, or introduction of additional feed material. Alternatively, a shell and tube reactor provided with a heat transfer medium may be used.

In preferred embodiments, the Group VIII metal containing catalyst is employed in a fixed bed reactor, e.g., in the shape of a pipe or tube, where the reactants, typically in vapor form, are passed over or through the catalyst. Other reactors, such as fluid or ebullient bed reactors, can be employed. In some instances, a hydrogenolysis catalyst may be used in conjunction with an inert material to regulate the pressure drop of the reactant stream through the catalyst bed and the contact time of the reactant compounds with the catalyst particles.

The hydrogenolysis process may be operated in a vapor phase, or a mixed vapor/liquid phase regime. The mixed vapor/liquid phase regime is where the reactant mixture, at the reactor conditions, is below the dew point temperature. The hydrogenolysis reaction may change from a mixed vapor/liquid phase to a fully vapor phase reaction, as the reaction proceeds down the reactor. The mixed phase hydrogenolysis may also be conducted in other types of reactors, or within a combination of different reactors, for example in a slurry or stirred tank reactor with, or without, external circulation and optionally operated as a cascade or stirred tank, a loop reactor or a Sulzer mixer-reactor. The hydrogenolysis process may be conducted in batch, semi-continuous, or continuous mode. For industrial purposes, continuous mode of operation is the most efficient.

In some embodiments, the hydrogenolysis reactor may comprise other types of reactors, such as fluidized bed, spinning basket and buss loop, or heat exchanger reactors. A mixed vapor/liquid phase hydrogenolysis reaction can be conducted with co-flow or counterflow of the vapor, e.g., hydrogen, to the liquid feed stream, in a bubble reactor. Trickle bed reactors may also be used.

The reduction of ethyl acetate to produce ethanol, e.g., in the hydrogenolysis reactor, is typically conducted at elevated temperatures from 125° C. to 350° C., e.g., from 180° C. to 345° C., from 225° C. to 310° C., or from 290° C. to 305° C. Reaction temperatures greater than 240° C., or greater than 260° C., may increase conversion of ethyl acetate. Although not bound by theory, it is believed that reduced temperatures in the hydrogenolysis reactor of less than 275° C. may suppress the formation of heavy impurities such as alcohols and/or ketones. The pressure in the hydrogenolysis reactor may operate under high pressure of greater than 1000 kPa, e.g., greater than 3,000 kPa or greater than 5,000 kPa. In terms of ranges the pressure in the hydrogenolysis reaction may be from 700 to 8,500 kPa, e.g., from 1,500 to 7,000 kPa, or from 2,000 to 6,500 kPa. Pressure greater than 2,500 kPa may be more favorable for improving ethanol productivity and/or selectivity. The reactants may be fed to hydrogenolysis reactor at a gas hourly space velocity (GHSV) of greater than 500 hr⁻¹, e.g., greater than 1000 hr⁻¹, greater than 2500 hr⁻¹ or even greater than 5000 hr⁻¹. In terms of ranges the GHSV may range from 50 hr⁻¹ to 20,000 hr⁻¹, e.g., from 1000 hr⁻¹ to 10,000 hr⁻¹, or from 2000 hr⁻¹ to 7,000 hr⁻¹.

In particular, the reaction of ethyl acetate may achieve favorable conversion of ethyl acetate and favorable selectivity and productivity to ethanol. For purposes of the present invention, the term “conversion” refers to the amount of ethyl acetate in the feed that is converted to a compound other than ethyl acetate. Conversion is expressed as a mole percentage based on ethyl acetate in the feed. The conversion may be at least 25%, e.g., at least 35%, at least 45%. In terms of ranges, the conversion of ethyl acetate may range from 25 to 95%, e.g., from 35 to 95% or from 45 to 90%. Although catalysts and reaction conditions that have high conversions may be possible, such as greater than 90% or greater than 95%, in some embodiments a low conversion may be acceptable at high selectivity for ethanol and low selectivity for ethers. Compensating for low conversion by appropriate recycle streams or use of larger reactors may be easier than compensating for poor selectivity to ethanol and/or ether.

Selectivity is expressed as a mole percent based on converted ethyl acetate. It should be understood that each compound converted from ethyl acetate has an independent selectivity and that selectivity is independent from conversion. For example, if 90 mole % of the converted ethyl acetate is converted to ethanol, we refer to the ethanol selectivity as 90%. The selectivity to ethanol is preferably at least 80%, e.g., at least 90% or at least 95%.

The term “productivity,” as used herein, refers to the grams of a specified product, e.g., ethanol, formed during the hydrogenolysis, based on the kilograms of catalyst used per hour. A productivity of at least 100 grams of ethanol per kilogram of catalyst per hour, e.g., at least 500 grams of ethanol per kilogram of catalyst per hour or at least 1,000 grams of ethanol per kilogram of catalyst per hour, is preferred. In terms of ranges, the productivity preferably is from 100 to 3,000 grams of ethanol per kilogram of catalyst per hour, e.g., from 400 to 2,500 grams of ethanol per kilogram of catalyst per hour or from 600 to 2,000 grams of ethanol per kilogram of catalyst per hour.

### Feed Stream

The ethyl acetate that is fed to the reactor may be obtained from any suitable source, including ethyl acetate produced by direct hydrogenation of acetic acid, ethanol dehydrogenation, or esterification of acetic acid and ethanol. Pure ethyl acetate may be used, but ethyl acetate that contains minor amounts of impurities, such as acetic acid, ethanol, acetaldehyde, and water, may also be used. In general, a suitable ethyl acetate feed stream may be enriched in ethyl acetate and contain from 90 to 99.9 wt. % ethyl acetate, and contain less than 5 wt. % ethanol and/or water. The feed stream may be substantially free of acetic acid. In addition, the feed stream to the hydrogenolysis reactor may be substantially free of diethyl ether. Direct hydrogenation of acetic acid may use a suitable hydrogenation catalyst as described in U.S. Pat. No. 7,820,852, the entire contents and disclosure of which is hereby incorporated by reference. Ethanol dehydrogenated may use a ruthenium carbon catalyst in the absence of acetic acid, as described in U.S. Pat. No. 6,809,217, the entire contents and disclosure of which is hereby incorporated by reference. Esterification of acetic acid and ethanol may use a suitable acidic catalyst as described in as described in U.S. Pat. No. 6,768,021, the entire contents and disclosure of which is hereby incorporated by reference.

In one exemplary embodiment, the present invention comprises producing ethanol from acetic acid by esterifying the acetic acid to form an ester and reducing the ester to an alcohol. The embodiments of the present invention may also be integrated with methods for producing acetic acid and/or methods for producing ethanol. For example, acetic acid may be produced from methanol, and thus ethanol production according to embodiments of the present invention may be produced from methanol. In one embodiment, the present invention comprises producing ethanol from methanol by carbonylating the methanol to form acetic acid, esterifying the acetic acid to form an ester, and reducing the ester to form ethanol. In yet another embodiment, the present invention comprises producing methanol from syngas, carbonylating the methanol to form acetic acid, esterifying the acetic acid to form an ester, and reducing the ester to an alcohol, namely ethanol. In still another embodiment, the present invention comprises producing ethanol from a carbon source, such as coal, biomass, petroleum, or natural gas, by converting the carbon source to syngas, followed by converting the syngas to methanol, carbonylating the methanol to form acetic acid, esterifying the acetic acid to form an ester, and reducing the ester to an alcohol. In still another embodiment, the present invention comprises producing ethanol from a carbon source, such as coal, biomass, petroleum, or natural gas, by converting the carbon source to syngas, separating the syngas into a hydrogen stream and a carbon monoxide stream, carbonylating a methanol with the carbon monoxide stream to form acetic acid, esterifying the acetic acid to form an ester, and reducing the ester to an alcohol. In addition, the ester may be reduced with the hydrogen stream. Also, methanol may be produced from the syngas.

### Esterification Integration

The hydrogenolysis reaction may be integrated with the any of the dehydrogenation, hydrogenation, or esterification processes. In a preferred embodiment, hydrogenolysis may be integrated with an esterification process.

The esterification reactants, acids and alcohols, used in connection with the process of this invention may be derived from any suitable source including carbon source such as natural gas, petroleum, coal, biomass, and so forth. Acetic acid may be produced by several methods, including but not limited to, methanol carbonylation, acetaldehyde oxidation, ethane oxidation, oxidative fermentation, and anaerobic fermentation.

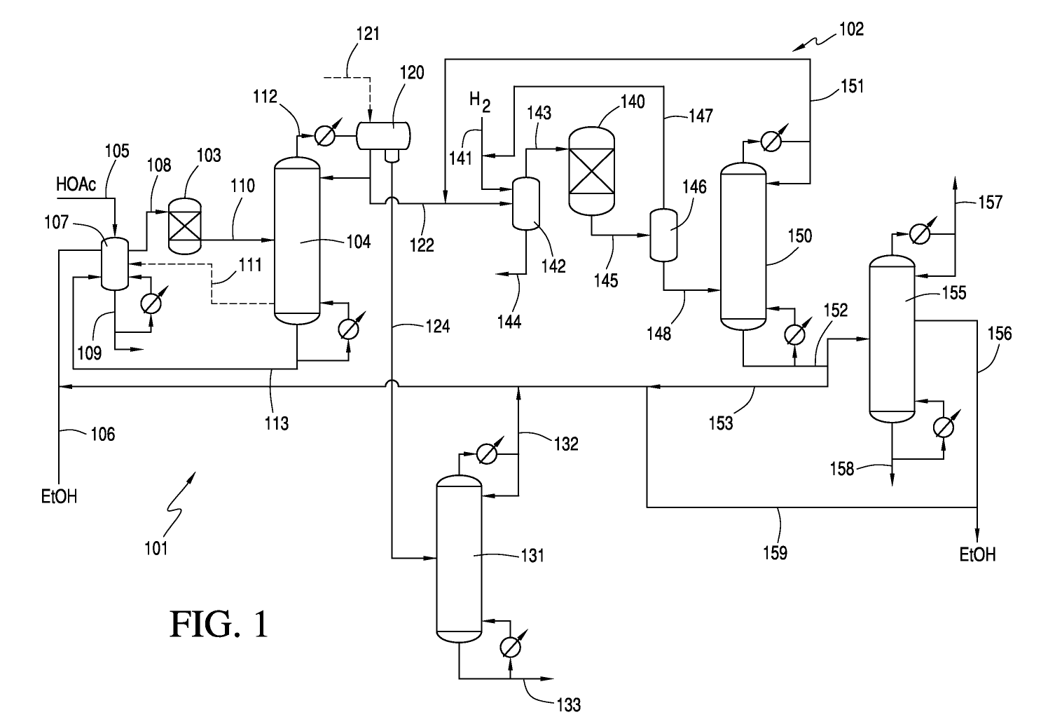

The process of the present invention comprises an esterification zone 101 and a hydrogenolysis zone 102 as shown in FIG. 1. Esterification may be carried out in either the liquid or vapor phase. The process may be operated continuously or batchwise. Liquid phase esterification of acetic acid and ethanol has an equilibrium constant, Kₓ, of about 4, while vapor phase esterification of acetic acid and ethanol has a higher equilibrium constant, Kₓ, of about 30 at 130° C.

The formation of the esterification product in the esterification equilibrium reaction may be enhanced by the presence of a catalyst. A variety of homogeneous or heterogeneous acid catalysts may also be employed within the scope of this invention. The catalyst should be stable at the desired reaction temperature. Suitable catalysts include, without limitation, sulfuric acid, sulfonic acid, alkyl sulfonic acids, and aromatic sulfonic acids. Alkyl sulfonic and aromatic sulfonic acids may include methane sulfonic acid, benzene sulfonic acid and p-toluene sulfonic acid. In one embodiment, an ion exchange resin, e.g., Amberlyst™ 15, Amberlyst™ 36, Amberlyst™ 70, or Purolite™ CT179, may be used. Sulfuric acid, acidic zeolites, or heteropoly acids can also be used within the scope of the invention.

In either the vapor or liquid phase reactions, although ethanol and acetic acid may be fed in equimolar amounts, in commercial ester production processes ethanol may be employed in excess molar amounts in the reaction mixture. In one aspect, because incomplete conversion of acetic acid in the esterification is less significant for purposes of the present invention, in some embodiments, it may be preferably to use an excess molar ratio of acetic acid. In one embodiment, the molar ratio of acetic acid to ethanol is greater than 1.01:1, e.g., greater than 1.05:1, greater than 1.2:1 or greater than 1.5:1. In terms of ranges, the molar ratio of acetic acid to ethanol may be from 1.01:1 to 5:1, e.g., from 1.01:1 to 3:1, from 1.05:1 to 3:1 or from 1.5:1 to 2.8:1. Without being bound by theory, the use of an excess molar amount of acetic acid, particularly under vapor phase esterification conditions, may desirably reduce formation of diethyl ether. This may advantageously reduce the amount of diethyl ether that is fed to the hydrogenolysis reactor. A molar ratio that is greater than 1.5:1 under vapor phase conditions, at reaction temperatures of less than 130° C., may result in substantially no formation of diethyl ether. Additionally, the use of excess acetic acid may allow for higher conversion rates of ethanol in the esterification reactor. In one embodiment, at least 75% of the ethanol fed to the esterification reactor is converted to ethyl acetate, e.g., at least 90% or at least 95%.

Vapor phase esterification may be carried out in a closely-coupled reactor 103 and distillation column 104 in the esterification zone 101 as shown in FIG. 1. Suitable reactors, in some embodiments, may include a variety of configurations using a fixed bed reactor or a fluidized bed reactor. In many embodiments of the present invention, an “adiabatic” reactor can be used; that is, there is little or no need for internal plumbing through the reaction zone to add or remove heat. In other embodiments, a radial flow reactor or reactors may be employed as the reactor, or a series of reactors may be employed with or without heat exchange, quenching, or introduction of additional feed material. Alternatively, a shell and tube reactor provided with a heat transfer medium may be used. In many cases, the reaction zone may be housed in a single vessel or in a series of vessels with heat exchangers therebetween. Reactor 103 may be a fixed-bed reactor and may comprise a heterogeneous catalyst.

In another embodiment, the reaction may be carried out in the vapor phase using a heterogeneous reactive distillation column. One or more reactors may be connected with the column.

Acetic acid feed stream in line 105 and ethanol feed stream in line 106, respectively, are fed to a vaporizer 107 to create a vapor feed stream in line 108 that is directed to reactor 103. In one embodiment, prior to feeding into vaporizer 107, the acetic acid feed stream and/or ethanol feed stream may be preheated. Vaporizer 107 may be fed with liquid reactants or vapor reactants, and preferably all the reactants are in the liquid phase. The acetic acid and ethanol may be vaporized at or near the reaction temperature. For reactions conducted in the vapor phase, the temperature should be controlled in the system such that it does not fall below the dew point of acetic acid. In one embodiment, the acetic acid may be vaporized at the boiling point of acetic acid at the particular pressure, and then the vaporized acetic acid may be further heated to the reactor inlet temperature. In another embodiment, the acetic acid is mixed with other gases before vaporizing, followed by heating the mixed vapors up to the reactor inlet temperature.

As shown in FIG. 1, acetic acid feed stream in line 105 may be fed to the top of vaporizer 107 and ethanol feed stream in line 106 may be fed at a point below the acetic acid feed stream point. The location of the feed points to vaporizer 107 may vary depending on the vessel configuration. Vaporizer 107 may be a vessel equipped with heat energy input sufficient to vaporize the liquid feed. The vessel may be jacketed, contain internal heating coils, or contain external thermosyphon, or forced circulation type reboilers. Optionally, lines 105 and 106 may be combined and jointly fed to vaporizer 107. Preferably, the vapor feed stream in line 108 is at a sufficient temperature to remain in the vapor phase. The temperature of the vapor feed stream in line 108 is preferably from 50° C. to 200° C., e.g., from 90° C. to 175° C. or from 100° C. to 170° C. In one embodiment, vapor feed stream 108 may be further preheated prior to being fed to reactor 103. The process may control the vapor-phase esterification reaction temperature by super-heating the vaporized feed in line 108 using a heat exchanger that is used to control the reactor inlet temperature.

Any feed that is not vaporized is removed from vaporizer 107 and may be recycled or discarded. In one embodiment, there may be a relatively small blowdown stream 109 that comprises heavy compounds that may be withdrawn from vaporizer 107. Blowdown stream 109 may be reboiled as necessary. The mass flow ratio of the vapor feed stream 108 to blowdown stream 109 may be greater than 5:1, e.g., greater than 50:1, or greater than 500:1. When ethanol from hydrogenolysis zone 102 is recycled to esterification zone 101, the ethanol may contain heavy compounds such as higher alcohols and/or higher acetates. These heavy compounds may buildup in the blowdown stream 109.

Although vaporizer 107 preferably comprises little or no acidic catalyst, due to the vaporization conditions, some acetic acid and ethanol may be esterified. Thus, vaporizer 107 may be a non-catalyzed reactor that produces ethyl acetate. Thus, vapor feed stream in line 108 in addition to containing acetic acid and ethanol, may also comprise minor amounts of ethyl acetate, e.g., in an amount of less than 15 wt. % based on the total weight of the vapor feed stream in line 108, e.g., less than 10 wt. % or less than 5 wt. %. In addition to the minor amounts of ethyl acetate, in one embodiment, vapor feed stream in line 108 may comprise a weight majority of acetic acid, e.g., at least 40 wt. %, at least 50 wt. % or at least 60 wt. %.

In one optional embodiment, there may be a liquid reactor (not shown) prior to vaporizer 107. The liquid reactor may contain a suitable acidic catalyst. Acetic acid feed stream in line 105 and ethanol feed stream in line 106 may be fed to the liquid reactor which produces an intermediate mixture that is vaporized. The additional feeds of acetic acid and ethanol may be fed with the intermediate mixture to vaporizer 107.

Vapor feed stream in line 108 is shown as being directed to the top of reactor 103 in FIG. 1, but in further embodiments, line 108 may be directed to the side, upper portion, or bottom of reactor 103. Reactor 103 contains the catalyst that is used in the esterification of acetic acid and ethanol. In one embodiment, one or more guard beds (not shown) may be used upstream of the reactor, optionally upstream of the vaporizer 107, to protect the catalyst from poisons or undesirable impurities contained in the feed or return/recycle streams. Such guard beds may be employed in the vapor or liquid streams. Suitable guard bed materials may include, for example, carbon, silica, alumina, ceramic, or resins. In one aspect, the guard bed media is functionalized, e.g., silver functionalized, to trap particular species such as sulfur or halogens.

The vapor-phase esterification reaction temperature is effected by the steady state composition and pressure, and typically may range from 50° C. to 200° C., e.g., from 80° C. to 190° C., from 125° C. to 175° C. The esterification process may be operated at atmospheric pressure but it is preferably operated at super-atmospheric pressure, e.g., from 105 to 700 kPa, from 110 to 350 kPa or from 120 to 300 kPa.

During the esterification process, an esterification product is withdrawn in vapor phase, preferably continuously, from reactor 103 via line 110. As shown in Table 1, the esterification product may comprise the following exemplary compositions.

TABLE 1
ESTERIFICATION PRODUCT

| Component | Conc. (wt. %) | Conc. (wt. %) | Conc. (wt. %) |
|---|---|---|---|
| Ethyl Acetate | 10 to 90 | 25 to 85 | 25 to 70 |
| Acetic Acid | 10 to 90 | 15 to 70 | 20 to 60 |
| Water | 0.5 to 30 | 1 to 20 | 1 to 15 |
| Ethanol | 0.01 to 10 | 0.01 to 5 | 0.01 to 4 |
| Diethyl ether | <0.1 | <0.01 | <0.001 |
| Acetaldehyde | <2 | <1 | <0.5 |
| Diethyl acetal | <1 | <0.1 | <0.05 |
| n-butyl acetate | <1 | <0.5 | <0.02 |
| 2-butyl acetate | <2 | <1 | <0.75 |
| Iso-propyl acetate | <1 | <0.5 | <0.1 |

The amounts indicated as less than (<) in the tables throughout the present specification may not be present and if present may be present in amounts greater than 0.0001 wt. %.

The trace impurities, such as n-butanol, 2-butanol, and/or iso-propanol, may be present in small amounts, if at all. Generally these other alcohols are also esterified to corresponding esters.

In one embodiment, esterification product in line 110 is fed directly to a first column 104, also referred to as an “azeotrope column.” In the embodiment shown in FIG. 1, line 110 is introduced in the lower part of first column 104. First column 104 may be a tray column having from 5 to 120 trays, e.g., from 15 to 80 trays or from 20 to 70 trays. In first column 104, acetic acid, a portion of the water, and other heavy components, if present, are withdrawn, preferably continuously, as first residue in line 113. First residue in line 113 may be reboiled as necessary to provide energy to drive the separation in column 104. First residue, or a portion thereof, in line 113 may be returned and/or recycled back to esterification zone 101 and fed to vaporizer 107. In addition, column 104 also recovers a first distillate in line 112. First distillate in line 112 may be condensed and further separated to recover a feed stream that is directed to hydrogenolysis zone 102 as described further herein.

In some embodiments, an optional vapor sidestream 111 from a lower portion of first column 104, which may be enriched in ethyl acetate as compared to first residue 113, may be withdrawn and returned to vaporizer 107. Optional vapor sidestream 111 may be withdrawn as a liquid or vapor and is preferably not reboiled in first column 104. Optional vapor sidestream 111 may provide energy necessary to drive vaporization in vaporizer 107 and thus reduce or eliminate the need for a reboiler.

The temperature of first column 104 at atmospheric pressure may vary. In one embodiment, the first residue exiting in line 113 preferably is at a temperature from 90° C. to 160° C., e.g., from 95° C. to 145° C. or from 100° C. to 140° C. The temperature of the first distillate exiting in line 112 preferably is from 60° C. to 125° C., e.g., from 85° C. to 110° C. or from 90° C. to 105° C. Column 104 may operate at an increased pressure, i.e., greater than atmospheric pressure. The pressure of column 104 may range from 105 to 510 kPa, from 110 to 475 kPa or from 120 to 375 kPa.

Exemplary components of the first distillate, and residue compositions for first column 104 are provided in Table 2 below. It should also be understood that the overhead stream and residue may also contain other components, not listed, such as components derived from the feed. For convenience, the residue of the first column may also be referred to as the “first residue.” The distillates or residues of the other columns may be referred to with similar numeric modifiers (second, third, etc.) in order to distinguish them from one another, but such modifiers should not be construed as requiring any particular separation order.

TABLE 2
FIRST COLUMN 104

| | Conc. (wt. %) | Conc. (wt. %) | Conc. (wt. %) |
|---|---|---|---|
| First Distillate | | | |
| Ethyl Acetate | 50 to 99.5 | 60 to 95 | 75 to 90 |
| Water | 1 to 50 | 1 to 20 | 3 to 15 |
| Ethanol | 0.01 to 10 | 0.01 to 5 | 0.5 to 4 |
| Acetic Acid | <0.5 | <0.02 | <0.01 |
| First Residue | | | |
| Acetic Acid | 50 to 99.5 | 60 to 95 | 75 to 90 |
| Ethyl Acetate | 0.01 to 20 | 0.5 to 15 | 1 to 10 |
| Water | 0.01 to 15 | 0.5 to 12 | 1 to 10 |
| Ethanol | 0.001 to 5 | 0.001 to 3 | 0.01 to 2 |

In another embodiment, the esterification may be carried in the liquid phase as described in U.S. Pat. Nos. 6,768,021, 6,765,110, and 4,481,146, the entire contents and disclosures of which are hereby incorporated by reference.

First distillate in line 112 from the vapor esterification in FIG. 1 may be biphasically separated in an overhead decanter 120. In some optional embodiments, a multi-stage extraction may be used. After esterification, the resulting vapors, e.g., esterification product, are collected at the top of the column as the first distillate and condensed. Condensing the first distillate may cause phase separation into a low density or lighter phase that is an organic phase rich in ethyl acetate and a more dense or heavier phase that is an aqueous phase rich in water. To further effectuate phasing, decanter 120 may be maintained a temperature from 0 to 40° C. In another embodiment, water may be added to decanter 120 to enhance phase separation via optional line 121. The optional water added to decanter 120 extracts ethanol from the organic phase thereby decreasing the water concentration in the organic phase. In other embodiments, the esterification product in the first distillate may have molar ratio of ethanol to ethyl acetate from 1:5 to 1:1.1, e.g., from 1:3 to 1:1.4, or from 1:2 to 1:1.25. A suitable molar ratio of ethanol to ethyl acetate to provide phasing may be 1.1:1.25. The low molar ratio of ethanol to ethyl acetate may also affect phasing. In addition, the low molar ratio of ethanol may also reduce the ethanol concentration in the organic phase and thus also reduce the water concentration in the organic phase.

Exemplary organic phase and aqueous phase compositions are provided in Table 3 below. These compositions may vary depending on the type of esterification reaction, e.g., liquid phase or vapor phase. Regardless of the type of esterification reaction, it is preferred that each phase contains very low concentrations of acetic acid, e.g., less than 600 wppm, e.g., less than 200 wppm or less than 50 wppm. In one embodiment, the organic phase comprises less than 6 wt. % ethanol and less than 5 wt. % water.

TABLE 3
OVERHEAD DECANTER 120

| | Conc. (wt. %) | Conc. (wt. %) | Conc. (wt. %) |
|---|---|---|---|
| Organic Phase | | | |
| Ethyl Acetate | 60 to 99.5 | 60 to 97 | 75 to 95 |
| Water | 0.01 to 10 | 0.5 to 8 | 0.5 to 5 |
| Ethanol | 0.01 to 10 | 0.5 to 6 | 0.5 to 5 |
| Diethyl acetal | <1 | <0.1 | <0.05 |
| C₃+ alcohols | <1 | <0.1 | <0.05 |
| Aqueous Phase | | | |
| Water | 60 to 99.5 | 60 to 97 | 75 to 95 |
| Ethyl Acetate | 0.01 to 30 | 0.5 to 25 | 1 to 15 |
| Ethanol | 0.01 to 20 | 0.1 to 15 | 0.5 to 10 |
| Diethyl acetal | <0.1 | <0.01 | <0.001 |
| C₃+ alcohols | <1 | <0.1 | <0.05 |

In some embodiments, an organic phase comprising ethyl acetate is removed from decanter 120 via line 122. As shown in FIG. 1, a portion of the organic phase from decanter 120 may also be refluxed via line 123 to the upper portion of first column 104. In one embodiment, the reflux ratio is from 0.5:1 to 1.2:1, e.g., from 0.6:1 to 1.1:1 or from 0.7:1 to 1:1. The remaining portion of organic phase in line 122, or an aliquot portion thereof, may be directly fed as the feed stream to hydrogenolysis zone 102 as shown in FIG. 1. In some embodiments, it may be preferred to preheat the organic phase directly fed to hydrogenolysis zone 102.

An aqueous phase comprising water is also removed from decanter 120 via line 124 and sent to recovery column 131, also referred to as the second column. Although a majority of the ethyl acetate is separated in the organic phase, a minor amount, e.g., less than 1%, or less than 0.75%, of the ethyl acetate in the decanter 120 may be withdrawn in the aqueous phase in line 124. In one embodiment, it is desirable to maximize ethyl acetate efficiency by recovering the ethyl acetate to be used as an azeotroping agent in first column 104 or to increase the ethyl acetate to ethanol in the hydrogenolysis zone 102. Optionally, a portion of the aqueous phase from the decanter 120 is purged and removed from the system.

In some embodiments, it may be desirable to further process the organic phase prior to entering hydrogenolysis zone 102. This may allow feeding a non-aliquot portion of the organic phase to hydrogenolysis zone 102. For example, the organic phase may be fed to a purification column (not shown) to reduce the ethanol and/or water concentrations and remove impurities. In another embodiment, the organic phase may be fed to a membrane separation unit or pervaporization (“pervap”) unit (not shown) to reduce water concentrations. In further embodiments of the present invention, the organic phase may be fed to a pervap unit and purification column in series. Further purification columns are described in U.S. application Ser. No. 13/299,730, filed on Nov. 18, 2011, the entire contents and disclosures of which are hereby incorporated by reference.

Recovery column 131 is operated to remove a significant portion of any organic content in aqueous phase in line 124 prior to purging the water. Recovery column 131 may be a tray or packed column. In one embodiment, recovery column 131 is a tray column having from 10 to 80 trays, e.g., from 20 to 75 trays or from 30 to 60 trays. Although the temperature and pressure of recovery column 131 may vary, when at atmospheric pressure the temperature of the overhead preferably is from 60° C. to 85° C., e.g., from 65° C. to 80° C. or from 70° C. to 75° C. The temperature at the base of recovery column 131 preferably is from 92° C. to 118° C., e.g., from 97° C. to 113° C. or from 100° C. to 108° C. In other embodiments, the pressure of recovery column 131 may be from 1 kPa to 300 kPa, e.g., from 10 kPa to 200 kPa or from 10 kPa to 150 kPa.

In one embodiment, any of the feeds to recovery column 131 may be at the top of the tower, i.e. near or into the reflux line. This keeps a sufficient loading on the trays such that the column operates as a stripping tower.

Exemplary second distillate and second residue compositions of recovery column 131 are provided in Table 4 below.

TABLE 4
RECOVERY COLUMN 131

| | Conc. (wt. %) | Conc. (wt. %) | Conc. (wt. %) |
|---|---|---|---|
| Second Distillate | | | |
| Ethyl Acetate | 20 to 80 | 35 to 75 | 40 to 55 |
| Water | 5 to 50 | 10 to 40 | 10 to 35 |
| Ethanol | 5 to 50 | 10 to 40 | 10 to 35 |
| C₃+ Acetates | <1 | <0.1 | <0.01 |
| C₃+ alcohols/ketones | <1 | <0.5 | <0.2 |
| Second Residue | | | |
| Water | 85 to 99.9 | 90 to 99.9 | 97 to 99.9 |
| Ethyl Acetate | 0.001 to 15 | 0.001 to 5 | 0.01 to 2 |
| Ethanol | 0.001 to 15 | 0.001 to 5 | 0.01 to 2 |
| C₃+ Acetates | <1 | <0.1 | <0.01 |
| C₃+ alcohols/ketones | <1 | <0.05 | <0.01 |

The second distillate of recovery column 131 in line 132 may be condensed and refluxed, as necessary, to the top of recovery column 131. Depending on the composition of overhead in line 132, the overhead may be returned to vaporizer 107, first column 104, or co-fed with a portion of the organic phase in line 122 to hydrogenolysis zone 102. When the second distillate in line 132 is fed to hydrogenolysis zone 102, it is preferable to control the total concentration of water such that it is less than 8 wt. % based on the total feed to hydrogenolysis section, e.g., less than 5 wt. % or less than 3 wt. %. In addition, particularly when the stream is relatively small, a portion of the second distillate in line 132 may be purged.

The second residue of recovery column 131, which mainly comprises water, is withdrawn in line 133. The water in line 133 may be purged from the system and optionally sent to waste water treatment. In some embodiments, a portion of the water may be returned to decanter 120 to maintain a desired water concentration for separation, fed as an extractive agent to one or more columns in the system, or used to hydrolyze impurities such as diethyl acetal in the process.

### III. Hydrogenolysis

In general, the ethyl acetate produced by the esterification reaction zone 101 is fed to hydrogenolysis reaction zone 102. As described above, ethyl acetate may be further purified from the esterification product before being fed to hydrogenolysis reaction zone 102. Regardless of the purification method, the feed stream preferably comprises less than 5 wppm esterification catalyst, e.g., less than 1 wppm, or less than 0.1 wppm. In addition, although acetic acid may not be separated from the esterification product, the process preferably is controlled such that the feed stream comprises less than 1 wt. % acetic acid, e.g., less than 0.1 wt. %, or less than 0.01 wt. %, and less than 0.5 wt. % diethyl ether, e.g., less than 0.1 wt. %, or less than 0.01 wt. %.

The amount of ethanol and/or water, if any, in the feed stream depends on the purification of the feed stream as described above. Preferably, the feed stream comprises less than 6 wt. % ethanol, e.g., less than 5 wt. % or less than 2 wt. %. The feed stream may also comprises less than 8 wt. % water, e.g., less than 5 wt. % or less than 3 wt. %.

As shown in FIG. 1, the organic phase in line 122 is referred to as the feed stream. In one embodiment, the feed stream 122 and hydrogen via feed line 141 are separately introduced into a vaporizer 142 to create a vapor feed stream in line 143 that is directed to hydrogenolysis reactor 140. In one embodiment, lines 122 and 141 may be combined and jointly fed to vaporizer 142. A vapor feed stream in line 143 is withdrawn from vaporizer 142 and is preheated by passing through a heat exchanger. The temperature of the vapor feed stream in line 143 after passing through the heat exchanger is preferably from 100° C. to 350° C., e.g., from 200° C. to 325° C. or from 250° C. to 300° C. Vaporizer 142 preferably operates at a pressure from 700 to 8,500 kPa, e.g., from 1,500 to 7,000 kPa, or from 2,000 to 6,500 kPa. Any feed that is not vaporized is removed from vaporizer 142 as a blowdown stream 144. Blowdown stream 144 may be discarded from the hydrogenolysis zone 102.

Although vapor feed stream in line 143 is shown as being directed to the top of hydrogenolysis reactor 140, line 143 may be directed to the side, upper portion, or bottom of hydrogenolysis reactor 140. Hydrogenolysis reactor 140 contains the Group VIII metal containing catalyst as described herein.

Hydrogen fed to hydrogenolysis reactor 140 may be obtained from syngas. In addition, hydrogen may also originate from a variety of other chemical processes, including ethylene crackers, styrene manufacturing, and catalytic reforming. Commercial processes for purposeful generation of hydrogen include autothermal reforming, steam reforming and partial oxidation of feedstocks such as natural gas, coal, coke, deasphalter bottoms, refinery residues and biomass. Hydrogen may also be produced by electrolysis of water. In one embodiment, the hydrogen is substantially pure and contains less than 10 mol.% carbon monoxide and/or carbon dioxide, e.g., less than 5 mol.% or less than 2 mol.%.

A crude ethanol product is preferably withdrawn continuously from hydrogenolysis reactor 140 via line 145. Any water in feed stream may pass through the hydrogenolysis reactor and be present in a similar amount in the crude ethanol product. The composition of the crude ethanol product may vary depending on the feed stream, conversion, and selectivity. Exemplary crude ethanol products, excluding hydrogen and other gases such as methane, ethane, carbon monoxide and/or carbon dioxide, are shown in Table 5 below.

TABLE 5
CRUDE ETHANOL PRODUCT

| Component | Conc. (wt. %) | Conc. (wt. %) | Conc. (wt. %) |
|---|---|---|---|
| Ethanol | 35 to 95 | 40 to 85 | 50 to 80 |
| Ethyl Acetate | 0.5 to 40 | 1 to 30 | 1 to 25 |
| Water | 0.001 to 10 | 0.001 to 5 | 0.001 to 3 |
| Aldehyde | <2 | 0.001 to 1.5 | 0.01 to 1 |
| Acetic Acid | <0.5 | <0.01 | <0.001 |
| Diethyl acetal | <1 | <0.1 | <0.05 |
| Diethyl ether | <0.5 | <0.1 | <0.05 |
| n-butanol | <1 | <0.5 | <0.1 |
| 2-butanol | 0.01 to 2 | 0.05 to 1.5 | 0.1 to 1 |
| Iso-propanol | <1 | <0.1 | <0.05 |
| Acetone | <1 | <0.5 | <0.1 |
| Heavies | <1 | <0.5 | <0.1 |
| Carbon Gases | 0.1 to 10 | 0.01 to 5 | 0.01 to 3 |

The crude ethanol product may have less than 0.5 wt. % ether, e.g., less than 0.3 wt. % or less than 0.1 wt. %. In one embodiment, the crude ethanol product may have an ether concentration from 0 to 0.5 wt. %, e.g., from 0.0001 to 0.5 wt. % or from 0.05 to 0.3 wt. %.

Heavies in Table 5 include organic compounds that have a larger molecular weight than ethanol, such as n-butyl acetate, sec-butyl acetate, ethyl butyrate, isopropyl acetate, 2-methyl-1-propanol, etc. Other acetates, aldehydes, and/or ketones may also be encompassed by heavies. The carbon gases refers to any carbon containing compound that is a gas at standard temperature and pressure, such as carbon monoxide, carbon dioxide, methane, ethane, etc. In one embodiment, the hydrogenolysis reaction is controlled to maintain low impurity concentrations of acetone, n-butanol, and 2-butanol.

The crude ethanol product in line 145 may be condensed and fed to a separator 146, which, in turn, provides a vapor stream 147 and a liquid stream 148. In some embodiments, separator 146 may comprise a flasher or a knockout pot. Also multiple separators may be used in series or in parallel. For example, multiple separators may be used in series, with each subsequent separator operating at a lower temperature and/or pressure. Although one separator 146 is shown, there may be multiple separators in some embodiments of the present invention. The separator 146 may operate at a temperature from 20° C. to 250° C., e.g., from 30° C. to 225° C. or from 60° C. to 200° C. The pressure of separator 146 may be greater than 1000 kPa, e.g., greater than 3,000 kPa or greater than 5,000 kPa. In terms of ranges the pressure in the separator may be from 700 to 8,500 kPa, e.g., from 1,500 to 7,000 kPa, or from 2,000 to 6,500 kPa.

Vapor stream 147 exiting separator 146 may comprise hydrogen, carbon monoxide, carbon dioxide, and hydrocarbons, and may be purged and/or returned to hydrogenolysis reactor 140. In some embodiments, the returned vapor stream 147 may be compressed before being combined with hydrogen feed 141. Vapor stream 147 may comprise inert gases, such as nitrogen, or nitrogen may be fed to vapor stream 147 to increase molecular weight for improved polytropic compression requirements. Vapor stream 147 may be combined with the hydrogen feed 141 and co-fed to vaporizer 142.

In FIG. 1, the liquid stream 148 from separator 146 is withdrawn and pumped to the side of a third distillation column 150, also referred to as a “light ends column,” to yield a third distillate in line 151 comprising ethyl acetate and a third residue in line 152 comprising ethanol. Preferably the distillation column operates to maintain a low concentration of ethyl acetate in the residue, e.g., less than 1 wt. %, less than 0.1 wt. % or less than 0.01 wt. %. The distillate of column 150 preferably is refluxed at a ratio sufficient to maintain low concentrations of ethyl acetate in the residue and minimize ethanol concentrations in the distillate, and reflux ratio may vary from 30:1 to 1:30, e.g., from 10:1 to 1:10 or from 5:1 to 1:5.

Distillation column 150 may be a tray column or packed column. In one embodiment, distillation column 150 is a tray column having from 5 to 110 trays, e.g., from 15 to 90 trays or from 20 to 80 trays. Distillation column 150 operates at a pressure ranging from 20 kPa to 500 kPa, e.g., from 50 kPa to 300 kPa or from 80 kPa to 200 kPa. Without being bound by theory, lower pressures of less than 100 kPa or less than 70 kPa, may further enhance separation of liquid stream 148. Although the temperature of distillation column 150 may vary, when at atmospheric pressure, the temperature of the distillate exiting in line 151 preferably is from 40° C. to 90° C., e.g., from 45° C. to 85° C. or from 50° C. to 80° C. The temperature of the residue exiting in line 152 preferably is from 45° C. to 95° C., e.g., from 50° C. to 90° C. or from 60° C. to 85° C.

Exemplary compositions of the third column 150 are shown in Table 6 below. It should be understood that the distillate and residue may also contain other components, not listed in Table 6.

TABLE 6
THIRD COLUMN 150 (FIG. 1)

| | Conc. (wt. %) | Conc. (wt. %) | Conc. (wt. %) |
|---|---|---|---|
| Third Distillate | | | |
| Ethyl Acetate | 20 to 80 | 25 to 75 | 30 to 70 |
| Ethanol | 0.01 to 45 | 1 to 35 | 2 to 30 |
| Water | <10 | <5 | <3 |
| Acetaldehyde | 0.01 to 30 | 0.1 to 20 | 1 to 10 |
| Diethyl Ether | <1 | <0.5 | <0.1 |
| Isopropanol | 0.001 to 0.5 | 0.001 to 0.1 | 0.001 to 0.05 |
| Acetone | 0.001 to 3 | 0.001 to 1 | 0.001 to 0.5 |
| Diethyl acetal | 0.001 to 3 | 0.001 to 1 | 0.01 to 0.5 |
| Carbon Gases | 0.001 to 2 | 0.001 to 1 | 0.001 to 0.5 |
| Third Residue | | | |
| Ethanol | 80 to 99.5 | 85 to 99.5 | 90 to 99.5 |
| Water | <20 | 0.001 to 15 | 0.01 to 10 |
| Ethyl Acetate | <0.01 | <0.001 | <0.0001 |
| Isopropanol | 0.001 to 3 | 0.001 to 1 | 0.001 to 0.5 |
| Acetone | 0.001 to 3 | 0.001 to 1 | 0.001 to 0.5 |
| Diethyl acetal | 0.001 to 3 | 0.001 to 1 | 0.01 to 0.5 |
| 2-butanol | 0.001 to 3 | 0.01 to 1 | 0.01 to 0.5 |
| n-butanol | <1 | <0.5 | <0.1 |
| Diethyl Ether | <1 | <0.5 | <0.1 |
| Heavies | <1 | <0.5 | <0.1 |

Without being bound by theory, the presence of acetaldehyde in the crude ethanol product from the hydrogenolysis reactor may produce several different impurities. The heavy impurities, such as higher alcohols, may build up in the third residue. In particular, 2-butanol has been found to be an impurity in this process. The weight ratio of 2-butanol to n-butanol in the third residue may be greater than 2:1, e.g., greater than 3:1 or greater than 5:1. Depending on the intended use of ethanol, these impurities may be of less significance. However, when a purer ethanol product is desired, a portion of third residue may be further separated in a finishing column 155 as described below.

In one embodiment, third distillate in line 151 may be returned, directly or indirectly, to hydrogenolysis reactor 140. When hydrogenolysis reactor 140 operates at a lower ethyl acetate conversion, e.g. less than 90% conversion, less than 85% conversion or less than 70% conversion, it may be possible to recycle ethyl acetate back to hydrogenolysis reactor 140. Third distillate in line 151 is condensed and combined with the feed stream and co-fed to vaporizer 142. This produces a distillate having a molar ratio of ethanol to ethyl acetate, of approximately 1:1. Advantageously, this embodiment may avoid recycling ethanol through hydrogenolysis reactor 140 that may lead to capacity restraints and additional capital costs. When returning third distillate to hydrogenolysis reactor 140, it is preferred to operate column 150 with a design and under conditions that minimize the ethanol to ethyl acetate ratio, e.g., distillation trays and/or reflux ratio.

In one embodiment, third distillate in line 151 may comprise other organic compounds such as aldehydes. Recycling the aldehydes to esterification reactor 103, may cause aldol condensation and result in the production of other byproducts. However, recycling a third distillate in line 151 that contains aldehydes to hydrogenolysis reactor 140 tends to produce additional ethanol.

Third residue in line 152 may be withdrawn as the product. In one embodiment, shown in FIG. 1, a portion of third residue in line 152 is separated into an ethanol return stream 153. Ethanol return stream 153 is fed to esterification zone 101. When reducing ethyl acetate in the presence of hydrogen, two moles of ethanol are formed. Thus, it may be feasible to return a portion of the ethanol to the esterification to produce additional ethyl acetate while still producing ethanol product.

Because ethanol return stream 153 is deficient in ethyl acetate for the purposes of azeotroping water in the overhead of first column 104, it may be necessary to combine ethanol return stream 153 with at least one ethyl acetate containing stream from the esterification separation processes. This will allow an azeotrope agent to be added to first column 104, as shown in FIG. 1. In some embodiments, the azeotrope agent may be directly added by passing through vaporizer 107 and reactor 103. For example, as shown in FIG. 1, second distillate 132 of recovery column 131 may be combined with the ethanol return stream 153. In other embodiments, the azeotrope agent may be added directly from an outside source to first column 104.

In an optional embodiment, although not shown, third distillate in line 151 may be returned, directly or indirectly, to esterification zone 101. Third distillate in line 151 may be combined with either the acetic acid feed stream in line 105 or ethanol feed stream in line 106. When third distillate 151 is returned to esterification reactor 103, it may be possible to return a relatively larger amount of ethanol. Optionally, third distillate in line 151 may be split and a portion may be fed to esterification reactor 103 and another portion to first column 104. Without being bound by theory, this also allows third column to operate under less stringent conditions, e.g., with a lower reflux ratio. In addition, when an appreciable amount of alcohols having at least 4 carbons, such as n-butanol and/or 2-butanol, are produced through side reactions in the hydrogenolysis reactor 140, it is preferred not to return these higher alcohols to the esterification step as the higher alcohols may react with acetic acid leading to a buildup of higher acetates in the process.

Third distillate in line 151 may have a higher ethanol to ethyl acetate ratio when directing this stream to esterification zone 101 as compared to the ethanol to ethyl acetate ratio when recycling back to hydrogenolysis zone 102. The additional ethyl acetate from third distillation column 150 may provide for an azeotrope agent to first column 104. In addition, the ethanol in the third distillate may be used to further esterify the acetic acid. Thus is may not be necessary to recycle any of the third residue in line 152 is returned to esterification zone.

In some embodiments, it may be necessary to further treat the third residue to remove additional heavy compounds such as higher alcohols and any light components from the ethanol. As shown in FIG. 1, there is provided a finishing column 155, also referred to as a “fourth column.” Third residue in line 152 is fed to a lower portion of fourth column 155. Fourth column 155 produces an ethanol sidestream in line 156, a fourth distillate in line 157 and a fourth residue in line 158. Preferably ethanol sidestream 156 is the largest stream withdrawn from fourth column 155 and is withdrawn at a point above the feed point of the third residue in line 152. In one embodiment the relative flow ratios of sidestream to residue is greater than 50:1, e.g., greater than 100:1 or greater than 150:1.

Ethanol sidestream 156 preferably comprises at least 90% ethanol, e.g., at least 92% ethanol and a least 95% ethanol. Depending on the amount of water fed to hydrogenolysis reactor 140, the water concentration in ethanol sidestream 156 may be less than 10 wt. %, e.g., less than 5 wt. % or less than 1 wt. %. In addition, the amount of other impurities, in particular diethyl acetal and 2-butanol, are preferably less than 0.05 wt. %, e.g., less than 0.03 wt. % or less than 0.01 wt. %. The fourth distillate in line 157 preferably comprises a weight majority of the diethyl acetal fed to fourth column 155. In addition, other light components, such as acetaldehyde and/or ethyl acetate may also concentrate in the fourth distillate. The fourth residue in line 158 preferably comprises a weight majority of the 2-butanol fed to fourth column 155. Heavier alcohols may also concentrate in the fourth residue in line 158.

Fourth column 155 may be a tray column or packed column. In one embodiment, Fourth column 155 is a tray column having from 10 to 100 trays, e.g., from 20 to 80 trays or from 30 to 70 trays. Fourth column 155 operates at a pressure ranging from 1 kPa to 510 kPa, e.g., from 10 kPa to 450 kPa or from 50 kPa to 350 kPa. Although the temperature of fourth column 155 may vary, the temperature of the residue exiting in line 158 preferably is from 70° C. to 105° C., e.g., from 70° C. to 100° C. or from 75° C. to 95° C. The temperature of the fourth distillate exiting in line 157 preferably is from 50° C. to 90° C., e.g., from 55° C. to 85° C. or from 65° C. to 80° C. Ethanol sidestream 156 is preferably withdrawn at the boiling point of ethanol, about 78° C. at atmospheric pressure. A portion of ethanol sidestream 156 in line 159 may be returned to esterification zone 101. In one embodiment, less than half of the ethanol sidestream 156 is returned via line 159. Returning ethanol in line 159 may reduce the amount of heavy compounds that are returned to esterification zone 101.

In some embodiments, a portion of the fourth residue, sidestream or fourth distillate may be dehydrated to form aliphatic alkenes. In one embodiment, the 2-butanol in the fourth residue may be dehydrated to 2-butene. In another embodiment, the 2-butanol in the fourth residue stream may be recovered in a separate system.

In one embodiment, instead of purging the fourth distillate in line 157 or the fourth residue in line 158, a portion thereof may be fed to vaporizer 107. Heavy ends compounds may be removed in the blowdown stream 109.

The ethanol product may contain small concentrations of water. For some ethanol applications, in particular for fuel applications, it may be desirable to further reduce the water concentration. Suitable water separation units may include an adsorption unit, one or more membranes, molecular sieves, extractive distillation units, or a combination thereof. Suitable adsorption units include pressure swing adsorption (PSA) units and thermal swing adsorption (TSA) units.

The columns shown in the figures may comprise any distillation column capable of performing the desired separation and/or purification. For example, unless described otherwise, the columns may be tray columns having from 1 to 150 trays, e.g., from 10 to 100 trays, from 20 to 95 trays or from 30 to 75 trays. The trays may be sieve trays, fixed valve trays, movable valve trays, or any other suitable design known in the art. In other embodiments, a packed column may be used. For packed columns, structured packing or random packing may be employed. The trays or packing may be arranged in one continuous column or may be arranged in two or more columns such that the vapor from the first section enters the second section while the liquid from the second section enters the first section, etc.

The associated condensers and liquid separation vessels that may be employed with each of the distillation columns may be of any conventional design and are simplified in the figures. Heat may be supplied to the base of each column or to a circulating bottom stream through a heat exchanger or reboiler. Other types of reboilers, such as internal reboilers, may also be used. The heat that is provided to the reboilers may be derived from any heat generated during the process that is integrated with the reboilers or from an external source such as another heat generating chemical process or a boiler. Although one reactor and one flasher are shown in the figures, additional reactors, flashers, condensers, heating elements, and other components may be used in various embodiments of the present invention. As will be recognized by those skilled in the art, various condensers, pumps, compressors, reboilers, drums, valves, connectors, separation vessels, etc., normally employed in carrying out chemical processes may also be combined and employed in the processes of the present invention.

The temperatures and pressures employed in the columns may vary. Temperatures within the various zones will normally range between the boiling points of the composition removed as the distillate and the composition removed as the residue. As will be recognized by those skilled in the art, the temperature at a given location in an operating distillation column is dependent on the composition of the material at that location and the pressure of column. In addition, feed rates may vary depending on the size of the production process and, if described, may be generically referred to in terms of feed weight ratios.

For purposes of the present invention, exemplary ethanol compositional ranges are provided below in Table 7. Depending on the application of the ethanol, one or more of the other organic impurities listed in Table 7 may be present.

TABLE 7
FINISHED ETHANOL COMPOSITIONS

| Component | Conc. (wt. %) | Conc. (wt. %) | Conc. (wt. %) |
|---|---|---|---|
| Ethanol | 75 to 99.9 | 88 to 99.5 | 90 to 96 |
| Water | <12 | 0.01 to 7.5 | 0.5 to 5 |
| Acetic Acid | <0.1 | <0.01 | <0.005 |
| Ethyl Acetate | <0.1 | <0.01 | <0.005 |
| Isopropanol | <0.5 | <0.1 | <0.05 |
| Diethyl Acetal | <0.5 | <0.1 | <0.05 |
| Diethyl ether | <0.5 | 0.0001 to 0.5 | 0.0001 to 0.1 |
| n-butanol | <0.5 | <0.1 | <0.05 |
| 2-butanol | <2 | <0.5 | <0.1 |
| Acetone | <0.5 | <0.1 | <0.05 |

In one embodiment, the recovered ethanol may have a composition that is from 92 wt. % to 97 wt. % ethanol, 3 wt. % to 8 wt. % water, 0.01 wt. % to 0.2 wt. % 2-butanol, and 0.02 wt. % to 0.08 wt. % isopropanol. The amount of 2-butanol may be greater than isopropanol. Preferably, other than 2-butanol and isopropanol, the recovered ethanol comprises less than 1 wt. % of one or more organic impurities selected from the group consisting of acetaldehyde, acetic acid, diethyl acetal, and ethyl acetate. The 2-butanol concentration in the ethanol sidestream may be reduced to an amount that is less than 0.01 wt. % when using a finishing column.

Ethanol produced by the embodiments of the present invention may be used in a variety of applications including fuels, solvents, chemical feedstocks, pharmaceutical products, cleansers, sanitizers, hydrogen transport or consumption. In fuel applications, ethanol may be blended with gasoline for motor vehicles such as automobiles, boats and small piston engine aircraft. In non-fuel applications, ethanol may be used as a solvent for toiletry and cosmetic preparations, detergents, disinfectants, coatings, inks, and pharmaceuticals. Ethanol may also be used as a processing solvent in manufacturing processes for medicinal products, food preparations, dyes, photochemicals and latex processing.

Ethanol may also be used as a chemical feedstock to make other chemicals such as vinegar, ethyl acrylate, ethyl acetate, ethylene, glycol ethers, ethylamines, ethyl benzene, aldehydes, butadiene, and higher alcohols, especially butanol. In another application, ethanol may be dehydrated to produce ethylene. Any known dehydration catalyst can be employed to dehydrate ethanol, such as those described in copending U.S. Pub. Nos. 2010/0030002 and 2010/0030001, the entire contents and disclosures of which are hereby incorporated by reference. A zeolite catalyst, for example, may be employed as the dehydration catalyst. Preferably, the zeolite has a pore diameter of at least about 0.6 nm, and preferred zeolites include dehydration catalysts selected from the group consisting of mordenites, ZSM-5, a zeolite X and a zeolite Y. Zeolite X is described, for example, in U.S. Pat. No. 2,882,244 and zeolite Y in U.S. Pat. No. 3,130,007, the entireties of which are hereby incorporated herein by reference.

In order that the invention disclosed herein may be more efficiently understood, examples are provided below. It should be understood that these examples are for illustrative purposes only and are not to be construed as limiting the invention in any manner.

### EXAMPLES

### Comparative Example A

### SiO₂—Al₂O₃—Pt(3)-Sn(1.8)

The support material was SiO₂-(0.05) Al₂O₃ KA160 catalyst support (SiO₂-(0.05)Al₂O₃, Sud Chemie, 14/30 mesh). The metal solutions were prepared by first adding Sn(Oac)₂ (0.204 g, 0.86 mmol) to a vial containing 4.75 ml of 1:1 diluted glacial acetic acid. The mixture was stirred for 15 min at room temperature, and then, 0.335 g (0.86 mmol) of solid Pt(NH₃)₄(NO₃)₂ were added. The mixture was stirred for another 15 min at room temperature, and then added drop wise to 5.0 g of dry support material in a 100 ml round-bottomed flask. The metal solution was stirred continuously until all of the Pt/Sn mixture had been added to the support material catalyst support while rotating the flask after every addition of metal solution. After completing the addition of the metal solution, the flask containing the impregnated catalyst was left standing at room temperature for two hours. The flask was then attached to a rotor evaporator (bath temperature 80° C.), and evacuated until dried while slowly rotating the flask. The material was then dried further overnight at 120° C., and then calcined using the following temperature program: 25°→160° C./ramp 5.0°/min; hold for 2.0 hours; 160° 500° C./ramp 2.0°/min; hold for 4 hours.

### Comparative Example B

### SiO₂—WO₃(10)-Pt(3)-Sn(1.8)

The WO₃-modified silica support was prepared as follows. A solution of 1.24 g (0.42 mmol) of (NH₄)₆H₂W₁₂O₄₀.n H₂O, (AMT) in deionized H₂O (14 ml) was added dropwise to 10.0 g of SiO₂ NPSGSS 61138catalyst support (SA=250 m²/g, 1/16 inch extrudates) in a 100 ml round-bottomed flask. The flask was left standing for two hours at room temperature, and then evacuated to dryness using a rotor evaporator (bath temperature 80° C.). The resulting material was dried at 120° C. overnight under circulation air, followed by calcination at 500° C. for 6 hours. All of the (light yellow) SiO₂—WO₃ material was then used for Pt/Sn metal impregnation using 0.6711 g (1.73 mmol) of Pt(NH₃)₄(NO₃)₂ and 0.4104 g (1.73 mmol) of Sn(OAc)₂ following the procedure described above for the SiO₂—PtₓSn₁₋ₓ materials. Yield: 12.10 g of dark grey 1/16 inch extrudates.

### Example 1

### SiO₂—CaSiO₃(5)-Pt(3)-Sn(1.8)

The material was prepared by first adding CaSiO₃ (Aldrich) to the SiO₂ catalyst support, followed by the addition of Pt/Sn as described previously. First, an aqueous suspension of CaSiO₃ (≦200 mesh) was prepared by adding 0.52 g of the solid to 13 ml of deionized H₂O, followed by the addition of 1.0 ml of colloidal SiO₂ (15 wt % solution, NALCO). The suspension was stirred for 2 h at room temperature and then added to 10.0 g of SiO₂ catalyst support (14/30 mesh) using incipient wetness technique. After standing for 2 hours, the material was evaporated to dryness, followed by drying at 120° C. overnight under circulating air and calcination at 500° C. for 6 hours. All of the SiO₂—CaSiO₃ material was then used for Pt/Sn metal impregnation using 0.6711 g (1.73 mmol) of Pt(NH₃)₄(NO₃)₂ and 0.4104 g (1.73 mmol) of Sn(OAc)₂ following the procedure described above for the SiO₂—PtₓSn₁₋ₓ materials. Yield: 11.21 g of dark grey material.

### Example 2

### Pt(1.09)Co(3.75)Sn(3.25)/CoSnWO₃/SiO₂

### A. Preparation of Modified Support: Co(3.75)Sn(3.25)WO₃(12)/SiO₂

A summary of the catalyst preparation protocol is provided in FIG. 1. A metal impregnation solution was prepared as follows. First, a solution of tin salt was prepared by adding 8.56 g (0.0414 mol) of SnC₂O₄ (solid) slowly into 41 g (0.328 mol) of 8M HNO₃ in a 300 ml beaker while stifling. 70 g of DI-H₂O was then added to further dilute the solution. 28 g (0.0962 mol) of Co(NO₃)₂.6H₂O solid was then added to the above solution with stirring. After the Co salt was completely dissolved, 19.47 g (0.079 mol W) of ammonium metatungstate (AMT) was added to the above solution. The mixture was then stirred at 400 rpm for another 5 minutes at room temperature.

The solution was then added to 120 g SiO₂ support in a one-liter round flask by using incipient wetness techniques to provide a uniform distribution on the support. After adding the solution, the material was evacuated to dryness using a rotary evaporator with bath temperature at 80° C. and vacuum at 72 mbar for 2 hours, followed by drying at 120° C. for 12 hours under circulating air and calcination at 600° C. for 8 hours. Temperature Program: Increase from room temperature to 160° C. at 3° C./min ramp, hold at 160° C. for 2 hours; increase from 160° C. to 600° C. at 3° C./min ramp, and hold at 600° C. for 8 hours.

### B. Impregnation of Modified Support: Pt(1.09)Co(3.75)Sn(3.25)/CoSnWO₃/SiO₂

A solution of tin salt was prepared by adding 6.28 g (0.0304 mol) of SnC₂O₄ (solid) slowly into 38.08 g (0.305 mol) of 8M HNO₃ in a 300 ml beaker while stirring. 13 g of DI-H₂O was added to further dilute the solution. 20.57 g (0.0707 mol) of Co(NO₃)₂.6H₂O was added to the solution with stifling. A solution of platinum oxalate was simultaneously prepared by diluting 11.72 g (6.08 mmol Pt) of platinum oxalate (Pt: 10.12 wt. %) with 15 g of DI-H₂O. The diluted platinum oxalate was added to above Co/Sn solution.

The resulting solution was then added to 100 g of the modified support pellets (CoSnWO₃/SiO₂) in a one-liter round flask by using incipient wetness techniques to provide a uniform distribution on the support. After adding the solution, the material was evacuated to dryness with a rotary evaporator at a bath temperature of 80° C. and vacuum at 72 mbar for 2 hours, followed by drying at 120° C. for 12 hours under circulating air and calcination at 350° C. for 8 hours. Temperature Program: increase from room temperature to 160° C. at 3° C./min ramp, hold at 160° C. for 2 hours, increase for 160° C. to 350° C. at 3° C./min ramp, hold at 350° C. for 8 hours. The impregnation solution was kept stifling during its addition to the support. The flask containing the support was continuously rotated during impregnation to ensure uniform distribution of the added liquid.

### Example 3

### Performance Tests

The catalysts of Examples 1-2 and Comparative Examples A and B were fed to a test unit using one of the following running conditions.

### Reactor System and Catalytic Testing Conditions.

The test unit comprised four independent tubular fixed bed reactor systems with common temperature control, pressure and gas and liquid feeds. The reactors were made of ⅜ inch (0.95 cm) 316 SS tubing, and were 12⅛ inches (30.8 cm) in length. The vaporizers were made of ⅜ inch (0.95 cm) 316 SS tubing and were 12⅜ inches (31.45 cm) in length. The reactors, vaporizers, and their respective effluent transfer lines were electrically heated (heat tape).

The reactor effluents were routed to chilled water condensers and knock-out pots. Condensed liquids were collected automatically, and then manually drained from the knock-out pots as needed. Non-condensed gases were passed through a manual back pressure regulator (BPR) and then scrubbed through water and vented to the fume hood. For each Example, 15 ml of catalyst (3 mm pellets) was loaded into reactor. Both inlet and outlet of the reactor were filled with glass beads (3 mm) to form the fixed bed. Ethyl acetate was used as the feed. The following running conditions for catalyst screening were used: T=275° C., P=300 psig (2068 kPag), [Feed of EtOAc]=0.16 ml/min (pump rate), [H₂]=513 L/min, [N₂]=0.1 L/min, and gas-hourly space velocity (GHSV)=1473 hr⁻¹.

The crude product was analyzed by gas chromatograph (Agilent GC Model 6850), equipped with a flame ionization detector. The GC analytical results of the liquid product effluent, excluding water, are provided below in Table 8.

TABLE 8

| | | Conver- | | | | | |
|---|---|---|---|---|---|---|---|
| | | sion of | Selectivity (%) | | | | |
| Examples | Catalysts | EtOAc | AcH | Ether | EtOH | HOAc | DEA |
| Compar- | Pt/Sn—SiO₂/ | 76.2 | 1 | 5.1 | 93.3 | 0 | 0 |
| ative A | Al₂O₃ | | | | | | |
| Compar- | Pt/Sn—SiO₂/ | 75.24 | 0.91 | 8.26 | 90.54 | 2.29 | 0.28 |
| ative B | WO₃ | | | | | | |
| Example | Pt/Sn—SiO₂/ | 27 | 0.95 | 0 | 98.96 | 5.46 | 0 |
| 1 | CaSiO₃ | | | | | | |
| Example | Pt/Sn/ | 42.7 | 0.92 | 0.23 | 98.74 | 0 | 0 |
| 2 | Co—SiO2/ | | | | | | |
| | Co/Sn/WO₃ | | | | | | |

The crude ethanol product for Comparative A contained over 3.9 wt. % diethyl ether and Comparative B contained over 6.2 wt. % diethyl ether. Thus, despite the higher conversions of ethyl acetate, more diethyl ether was produced. In contrast, the lower ether selectivities of Examples 1 and 2 resulted in low diethyl ether concentrations in the crude ethanol product. Example 2 contained less than 0.1 wt. % diethyl ether and Example 1 contained no diethyl ether.

While the invention has been described in detail, modifications within the spirit and scope of the invention will be readily apparent to those skilled in the art. All publications and references discussed above are incorporated herein by reference. In addition, it should be understood that aspects of the invention and portions of various embodiments and various features recited may be combined or interchanged either in whole or in part. In the foregoing descriptions of the various embodiments, those embodiments which refer to another embodiment may be appropriately combined with other embodiments as will be appreciated by one skilled in the art. Furthermore, those skilled in the art will appreciate that the foregoing description is by way of example only, and is not intended to limit the invention.

What is claimed is:

1. A process for producing ethanol comprising
contacting a feed stream comprising ethyl acetate with hydrogen in a reactor in the presence of a catalyst to produce a crude ethanol product that comprises less than 0.5 wt. % ether compounds, wherein the catalyst comprises tin, a Group VIII metal selected from the group consisting of palladium, platinum, and combinations thereof, and a support that comprises calcium, magnesium, tungsten, or molybdenum, provided that when the support comprises tungsten and/or molybdenum, the catalyst further comprises cobalt and/or the support further comprises cobalt and/or tin.

2. The process of claim 1, wherein the crude ethanol product comprises from 0.0001 to 0.5 wt. % diethyl ether.

3. The process of claim 1, wherein the support comprises a support material selected from the group consisting of silica, pyrogenic silica, and high purity silica.

4. The process of claim 3, wherein the support material is substantially free of alumina.

5. The process of claim 1, wherein the selectivity to ether compounds is less than 1%.

6. The process of claim 1, wherein the support comprises calcium oxide, calcium silicate, calcium metasilicate, magnesium oxide, magnesium silicate, or magnesium metasilicate.

7. The process of claim 1, wherein the support comprises from 1 to 25 wt. % calcium or magnesium, based on the total weight of the catalyst.

8. The process of claim 1, wherein the support comprises tungsten oxide, cobalt tungstate, molybdenum oxide, cobalt molybdate, or combinations thereof.

9. The process of claim 8, wherein the support is substantially free of tin tungstate.

10. The process of claim 1, wherein the support comprises from 5 to 30 wt. % tungsten or molybdenum, based on the total weight of the catalyst.

11. The process of claim 1, wherein the tin is present from 0.1 to 7.5 wt. %, based on the total weight of the catalyst.

12. The process of claim 1, wherein the Group VIII metal is present from 0.1 to 3 wt. %, based on the total weight of the catalyst.

13. The process of claim 1, wherein the support comprises tungsten and/or molybdenum, cobalt is present from 0.1 to 20 wt. %, based on the total weight of the catalyst.

14. The process of claim 1, wherein the support comprises tungsten, and/or molybdenum, and the support further comprises cobalt and tin.

15. The process of claim 1, further comprising separating ethyl acetate, acetic acid, and acetaldehyde from the crude ethanol mixture and recycling the separated compounds to the feed stream.

16. The process of claim 1, wherein the feed stream comprises from 90 to 99.9 wt. % ethyl acetate.

17. A method of producing ethanol comprising:
directing acetic acid and ethanol in a molar ratio of greater than 1.01:1 to a first reaction zone to produce a feed stream; and
reacting at least a portion of the feed stream with hydrogen in a second reaction zone to produce a crude ethanol product, wherein the second reaction zone contains a catalyst that comprises tin, a Group VIII metal selected from the group consisting of palladium and platinum, and a support that comprises calcium, magnesium, tungsten, or molybdenum, provided that when the support comprises tungsten and/or molybdenum, the catalyst further comprises cobalt and/or the support further comprises cobalt and/or tin.

18. The process of claim 17, wherein the crude ethanol product comprises less than 0.5 wt. % diethyl ether.

19. A method of producing ethanol comprising:
esterifying acetic acid and ethanol in a first reaction zone to produce a feed stream;
reacting at least a portion of the feed stream with hydrogen in a second reaction zone to produce a crude ethanol product comprising ethyl acetate, ethanol, and at least one alcohol having at least 4 carbon atoms, wherein the second reaction zone contains a catalyst that comprises tin, a Group VIII metal selected from the group consisting of palladium, and platinum, and a support that comprises calcium, magnesium, tungsten, or molybdenum, provided that when the support comprises tungsten and/or molybdenum, the catalyst further comprises cobalt and/or the support further comprises cobalt and/or tin;
separating at least a portion of the crude ethanol product in a first distillation column to yield a first distillate comprising ethyl acetate and a first residue comprising ethanol; and
separating at least a portion of the first residue in a second distillation column to yield an ethanol side stream and a second residue comprising the at least one alcohol having at least 4 carbon atoms.

20. The process of claim 19, wherein the crude ethanol product comprises less than 0.5 wt. % diethyl ether.
